(19) United States
(12) Patent Application Publication
(10) Pub. No.: US 2024/0343804 A1
(43) Pub. Date: Oct. 17, 2024

(54) ASSESSING AND TREATING MESOTHELIOMA

(71) Applicant: Mayo Foundation for Medical Education and Research, Rochester, MN (US)

(72) Inventors: Aaron S. Mansfield, Rochester, MN (US); Mitesh J. Borad, Paradise Valley, AZ (US); George Vasmatzis, Oronoco, MN (US); Farhad Kosari, Rochester, MN (US)

(21) Appl. No.: 18/294,747

(22) PCT Filed: Aug. 17, 2022
(86) PCT No.: PCT/US2022/040537

§ 371 Date: Feb. 2, 2024

Related U.S. Application Data
(60) Provisional application No. 63/292,722, filed on Dec. 22, 2021.
(60) Provisional application No. 63/234,619, filed on Aug. 18, 2021.

(30) Foreign Application Priority Data
Oct. 27, 2021 (GR) 20210100739

(51) Int. Cl. C07K 16/28; A61K 39/00; A61P 35/00; C12Q 1/6886

(52) U.S. Cl. CPC C07K 16/2818; A61P 35/00; C12Q 1/6886; A61K 2039/505; C12Q 2600/158

(57) ABSTRACT

This document provides methods and materials for assessing and/or treating mammals (e.g., humans) having mesothelioma (e.g., mesothelioma with a high junction burden). For example, methods and materials that can be used to determine whether or not a mammal having mesothelioma is likely to respond to a particular cancer treatment (e.g., immunotherapy with one or more immune checkpoint inhibitors) are provided. Methods and materials for treating a mammal (e.g., a human) having mesothelioma (e.g., mesothelioma with a high junction burden) where the treatment is selected based, at least in part, on whether or not the mammal is likely to respond to a particular cancer treatment (e.g., immunotherapy with one or more immune checkpoint inhibitors) also are provided.

### CROSS-REFERENCE TO RELATED APPLICATIONS

This application claims the benefit of U.S. Patent Application Ser. No. 63/234,619, filed on Aug. 18, 2021, Greek patent application No. 20210100739, filed on Oct. 27, 2021, and U.S. Patent Application Ser. No. 63/292,722, filed on Dec. 22, 2021. The disclosures of the prior applications are considered part of (and are incorporated by reference in) the disclosure of this application.

### STATEMENT REGARDING FEDERAL FUNDING

This invention was made with government support under CA251923 awarded by the National Institutes of Health. The government has certain rights in the invention.

### TECHNICAL FIELD

This document relates to methods and materials for assessing and/or treating mammals (e.g., humans) having mesothelioma (e.g., mesothelioma with a high junction burden). For example, methods and materials provided herein can be used to determine whether or not a mammal having mesothelioma is likely to respond to a particular cancer treatment (e.g., immunotherapy with one or more immune checkpoint inhibitors). This document also provides methods and materials for treating a mammal (e.g., a human) having mesothelioma (e.g., mesothelioma with a high junction burden) where the treatment is selected based, at least in part, on whether or not the mammal is likely to respond to a particular cancer treatment (e.g., immunotherapy with one or more immune checkpoint inhibitors).

### BACKGROUND INFORMATION

Mesothelioma is an aggressive cancer with an average life expectancy of 14 to 22 months. The 5-year relative survival rate for malignant pleural mesothelioma (based on people diagnosed with malignant pleural mesothelioma (MPM) between 2010 and 2016) was only about 10% (American Cancer Society, “Survival Rates for Mesothelioma,” Last Revised: Jan. 21, 2021).

Mesothelioma primarily arises as a result of the exposure to the carcinogen asbestos, although some cases develop after therapeutic radiation, or are inherited due to loss of function mutations in BRCA1 Associated Protein 1 (BAP1) (Carbone et al., CA Cancer J. Clin., 69:402-429 (2019)). Immunotherapy is a frontline treatment option for MPM with the approval of the PD-1 inhibitor nivolumab and the CTLA-4 inhibitor ipilimumab.

### SUMMARY

In some cases, this document provides methods and materials for determining whether or not a mammal (e.g., a human) having mesothelioma (e.g., mesothelioma with a high junction burden) is likely to respond a particular cancer treatment (e.g., immunotherapy with one or more immune checkpoint inhibitors). For example, a sample obtained from a mammal having mesothelioma can be assessed to determine if the mammal is likely to respond a particular cancer treatment (e.g., immunotherapy with one or more immune checkpoint inhibitors) based, at least in part, on the presence or absence of an elevated level of at least 6 (e.g., 6, 8, 14, 17, 24, or more) APP polypeptides in the sample. As used herein, an APP polypeptide refers to a polypeptide involved in the antigen processing and presentation pathway. Examples of APP polypeptides and nucleic acids encoding APP polypeptides are provided in Table 4. This document also provides methods and materials for treating a mammal (e.g., a human) having mesothelioma (e.g., mesothelioma with a high junction burden) where the treatment is selected based, at least in part, on whether or not the mammal is likely to respond a particular cancer treatment (e.g., immunotherapy with one or more immune checkpoint inhibitors). For example, a mammal (e.g., a human) having mesothelioma (e.g., mesothelioma with a high junction burden) can be administered one or more cancer treatments that are selected based, at least in part, on the presence or absence of an elevated level of at least 6 different APP polypeptides in a sample obtained from the mammal.

As demonstrated herein, the presence of an elevated level of at least 6 different APP polypeptides can indicate that a mammal (e.g., human) having mesothelioma with a high junction burden is likely to be responsive to a particular cancer treatment (e.g., immunotherapy with one or more immune checkpoint inhibitors). For example, mammals (e.g., humans) having a mesothelioma with a high junction burden and having the presence of an elevated level of polypeptides expressed by APP genes can exhibit improved survival (e.g., as compared to patients having a high junction burden and lacking the presence of an elevated level of polypeptides expressed by APP genes) when treated with immune checkpoint inhibitors.

Having the ability to identify a particular cancer treatment that a mammal (e.g., a human) is most likely to respond allows clinicians to provide an individualized approach in selected cancer treatments, thereby improving disease-free survival and/or overall survival and/or minimizing subjecting patients to ineffective treatments.

In general, one aspect of this document features methods for assessing a mammal having mesothelioma. The methods can include, or consist essentially of, (a) determining if mesothelioma cells of a mammal having mesothelioma with a high junction burden contain the presence or absence of an elevated level of at least 6 APP polypeptides, (b) classifying the mammal as being likely to respond to an immune checkpoint inhibitor if the presence is determined; and (c) classifying the mammal as not being likely to respond to the immune checkpoint inhibitor if the absence is determined. The mammal can be a human. A tissue sample including the mesothelioma cells can be used to determine if the mesothelioma cells contain the presence or absence. The tissue sample can be a pleural tissue sample. The high junction burden can include at least 17 junctions per megabase (mb). The mesothelioma can be a pleural mesothelioma. The APP polypeptides can include a PYCARD polypeptide, a HFE polypeptide, a HLA-DOA polypeptide, a HLA-DOB polypeptide, a TREM2 polypeptide, a TAPBPL polypeptide, a HLA-A polypeptide, a HLA-B polypeptide, a HLA-C polypeptide, a HLA-E polypeptide, a HLA-F polypeptide, a HLA-G polypeptide, a HLA-H polypeptide, a AZGP1 polypeptide, a B2M polypeptide, a TAP2 polypeptide, a ABCB9 polypeptide, a IDE polypeptide, a ERAP1 polypeptide, a ERAP2 polypeptide, a TAP1 polypeptide, a TAPBP polypeptide, a AP3B1 polypeptide, a AP3D1 polypeptide, a CD1A polypeptide, a CD1B polypeptide, a CD1C polypeptide, a CD1D polypeptide, a CD1E polypeptide, a ATG5 polypeptide, or a CD74 polypeptide. The immune checkpoint inhibitor can be pembrolizumab, nivolumab, cemiplimab, atezolizumab, avelumab, durvalumab, or ipilimumab. The method can include determining the presence. The method can include determining the absence. The presence or absence of the elevated level of at least one of the at least 6 APP polypeptides can be determined by measuring mRNA encoding the at least one of the at least 6 APP polypeptides. The presence or absence of the elevated level of the of the at least 6 APP polypeptides can be determined by measuring mRNA encoding the each of the at least 6 APP polypeptides.

In another aspect, this document features methods for treating a mammal having mesothelioma. The methods can include, or consist essentially of, (a) determining that mesothelioma cells of a mammal having mesothelioma with high junction burden contain the presence of an elevated level of at least 6 APP polypeptides; and (b) administering an immune checkpoint inhibitor to the mammal. The mammal can be a human. A tissue sample comprising the mesothelioma cells can be used to determine that the mesothelioma cells contain the presence. The tissue sample can be a pleural tissue sample. The high junction burden can include at least 17 junctions per mb. The mesothelioma can be a pleural mesothelioma. The APP polypeptides can include a PYCARD polypeptide, a HFE polypeptide, a HLA-DOA polypeptide, a HLA-DOB polypeptide, a TREM2 polypeptide, a TAPBPL polypeptide, a HLA-A polypeptide, a HLA-B polypeptide, a HLA-C polypeptide, a HLA-E polypeptide, a HLA-F polypeptide, a HLA-G polypeptide, a HLA-H polypeptide, a AZGP1 polypeptide, a B2M polypeptide, a TAP2 polypeptide, a ABCB9 polypeptide, a IDE polypeptide, a ERAP1 polypeptide, a ERAP2 polypeptide, a TAP1 polypeptide, a TAPBP polypeptide, a AP3B1 polypeptide, a AP3D1 polypeptide, a CD1A polypeptide, a CD1B polypeptide, a CD1C polypeptide, a CD1D polypeptide, a CD1E polypeptide, a ATG5 polypeptide, or a CD74 polypeptide. The immune checkpoint inhibitor can be pembrolizumab, nivolumab, cemiplimab, atezolizumab, avelumab, durvalumab, or ipilimumab. The method also can include administering to the mammal a cancer treatment selected from the group consisting of administering cisplatin, administering pemetrexed, administering carboplain, administering bevacizumab, administering vinorelbine, administering gemcitabine, administering ramucirumab, surgery, radiation therapy, tumor treatment fields, and cryotherapy. The presence of the elevated level of at least one of the at least 6 APP polypeptides can be determined by measuring mRNA encoding the at least one of the at least 6 APP polypeptides. The presence of the elevated level of the of said at the 6 APP polypeptides can be determined by measuring mRNA encoding the each of the at least 6 APP polypeptides.

In another aspect, this document features methods for treating a mammal having mesothelioma. The methods can include, or consist essentially of, administering an immune checkpoint inhibitor to a mammal having mesothelioma with high junction burden and identified as having an elevated level of at least 6 APP polypeptides in mesothelioma cells of the mammal. The mammal can be a human. A tissue sample including the mesothelioma cells was used to identify the mammal as having an elevated level of the at least 6 APP polypeptides. The tissue sample can be a pleural tissue sample. The high junction burden can include at least 17 junctions per mb. The mesothelioma can be a pleural mesothelioma. The APP polypeptides can include a PYCARD polypeptide, a HFE polypeptide, a HLA-DOA polypeptide, a HLA-DOB polypeptide, a TREM2 polypeptide, a TAPBPL polypeptide, a HLA-A polypeptide, a HLA-B polypeptide, a HLA-C polypeptide, a HLA-E polypeptide, a HLA-F polypeptide, a HLA-G polypeptide, a HLA-H polypeptide, a AZGP1 polypeptide, a B2M polypeptide, a TAP2 polypeptide, a ABCB9 polypeptide, a IDE polypeptide, a ERAP1 polypeptide, a ERAP2 polypeptide, a TAP1 polypeptide, a TAPBP polypeptide, a AP3B1 polypeptide, a AP3D1 polypeptide, a CD1A polypeptide, a CD1B polypeptide, a CD1C polypeptide, a CD1D polypeptide, a CD1E polypeptide, a ATG5 polypeptide, or a CD74 polypeptide. The immune checkpoint inhibitor can be pembrolizumab, nivolumab, cemiplimab, atezolizumab, avelumab, durvalumab, or ipilimumab. The method also can include administering to the mammal a cancer treatment selected from the group consisting of administering cisplatin, administering pemetrexed, administering carboplain, administering bevacizumab, administering vinorelbine, administering gemcitabine, administering ramucirumab, surgery, radiation therapy, tumor treatment fields, and cryotherapy.

In another aspect, this document features methods for treating a mammal having mesothelioma. The methods can include, or consist essentially of, (a) determining if a sample from a mammal having mesothelioma with high junction burden contains the absence of an elevated level of polypeptides expressed by at least 6 APP genes; and (b) administering a cancer treatment to the mammal, where the cancer treatment is not an immune checkpoint inhibitor. The mammal can be a human. A tissue sample including the mesothelioma cells can be used to determine that the mesothelioma cells contain the absence. The tissue sample can be a pleural tissue sample. The high junction burden can include at least 17 junctions per mb. The mesothelioma can be a pleural mesothelioma. The APP polypeptides can include a PYCARD polypeptide, a HFE polypeptide, a HLA-DOA polypeptide, a HLA-DOB polypeptide, a TREM2 polypeptide, a TAPBPL polypeptide, a HLA-A polypeptide, a HLA-B polypeptide, a HLA-C polypeptide, a HLA-E polypeptide, a HLA-F polypeptide, a HLA-G polypeptide, a HLA-H polypeptide, a AZGP1 polypeptide, a B2M polypeptide, a TAP2 polypeptide, a ABCB9 polypeptide, a IDE polypeptide, a ERAP1 polypeptide, a ERAP2 polypeptide, a TAP1 polypeptide, a TAPBP polypeptide, a AP3B1 polypeptide, a AP3D1 polypeptide, a CD1A polypeptide, a CD1B polypeptide, a CD1C polypeptide, a CD1D polypeptide, a CD1E polypeptide, a ATG5 polypeptide, or a CD74 polypeptide. The cancer treatment can include administering to the mammal a cancer drug selected from the group consisting of cisplatin, pemetrexed, carboplain, bevacizumab, vinorelbine, gemcitabine, and ramucirumab. The cancer treatment can include subjecting the mammal to a treatment selected from the group consisting of surgery, radiation therapy, tumor treatment fields, and cryotherapy. The absence of the elevated level of at least one of the at least 6 APP polypeptides is determined by measuring mRNA encoding the at least one of the at least 6 APP polypeptides. The absence of the elevated level of each of the at least 6 APP polypeptides is determined by measuring mRNA encoding the each of the at least 6 APP polypeptides.

In another aspect, this document features methods for treating a mammal having mesothelioma. The methods can include, or consist essentially of, administering cancer treatment to a mammal having mesothelioma with high junction burden and identified as lacking an elevated level of polypeptides expressed by at least 6 APP genes in a sample obtained from the mammal, where the cancer treatment is not an immune checkpoint inhibitor. The mammal can be a human. A tissue sample including the mesothelioma cells was used to identify the mammal as lacking an elevated level of the at least 6 APP polypeptides. The tissue sample can be a pleural tissue sample. The high junction burden can include at least 17 junctions per mb. The mesothelioma can be a pleural mesothelioma. The APP polypeptides can include a PYCARD polypeptide, a HFE polypeptide, a HLA-DOA polypeptide, a HLA-DOB polypeptide, a TREM2 polypeptide, a TAPBPL polypeptide, a HLA-A polypeptide, a HLA-B polypeptide, a HLA-C polypeptide, a HLA-E polypeptide, a HLA-F polypeptide, a HLA-G polypeptide, a HLA-H polypeptide, a AZGP1 polypeptide, a B2M polypeptide, a TAP2 polypeptide, a ABCB9 polypeptide, a IDE polypeptide, a ERAP1 polypeptide, a ERAP2 polypeptide, a TAP1 polypeptide, a TAPBP polypeptide, a AP3B1 polypeptide, a AP3D1 polypeptide, a CD1A polypeptide, a CD1B polypeptide, a CD1C polypeptide, a CD1D polypeptide, a CD1E polypeptide, a ATG5 polypeptide, or a CD74 polypeptide. The cancer treatment can include administering to the mammal a cancer drug selected from the group consisting of cisplatin, pemetrexed, carboplain, bevacizumab, vinorelbine, gemcitabine, and ramucirumab. The cancer treatment can include subjecting the mammal to a treatment selected from the group consisting of surgery, radiation therapy, tumor treatment fields, and cryotherapy.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings.

### DESCRIPTION OF THE DRAWINGS

FIGS. 1A-1B. FIG. 1A) Survival times of the study cohort. Representative responses of progression of disease (PD), stable disease (SD), partial response (PR), epithelioid (Epit), sarcomatoid (Sar, including mesenchymal), and mixed (Mix) histology, are shown. “+” designates alive at the last follow-up. FIG. 1B) Heatmap representing survival times, junction burden, antigen processing and presentation, and immune checkpoint markers. The lower bar represents best responses with PD, SD, and PR as per FIG. 1A. Light grey arrows point to two cases with high junction burdens, short survival times, and low expression of genes encoding polypeptides involved in the antigen processing and presentation (APP) pathway. On the contrary, dark grey arrows point to two cases with moderate junction burdens, long survival times, and robust expression of nucleic acid encoding APP polypeptides.

FIG. 2. Forest plots displaying the hazard ratios for junction burdens and overall survival associations in samples with high and low APP gene set expression, respectively in gene sets identified as significant.

FIGS. 3A-3D. FIG. 3A) A Kaplan Meier curve representing a survival model based on the interactions between “REGULATION OF APP OF PEPTIDE ANTIGEN” gene-set and junction burdens is shown. Both the interaction terms and the log-rank test were significant. FIG. 3B) The ROC curve representing APP and log 2 [junction burden] interactions (cyan) in predicting NSB and SB is shown for the REGULATION_OF_AP&P_OF_PEPTIDE_ANTIGEN gene set. FIG. 3C) ROC curves representing the accuracy of TIDE, IFNG, PD-L1, and CD8 models in predicting NSB and SB. FIG. 3D) ROC curves representing APP, log 2 [junction burden], CD8A, and the final model including APP/log 2 [junction burden] interactions and CD8A. The inlet is a boxplot and individual patient prediction values by the final model in NSB and SB categories.

FIG. 4. Overall survival times of patients with MPM from the 2019 cohort are shown. Circles, triangles, and squares represent epithelioid (Epit), sarcomatoid (Sar, including desmoplastic), and mixed or other (MixOtr) subtypes.

FIGS. 5A-5B. FIG. 5A) Kaplan Meier plot representing associations between the gene signature of Lopez-Rios et al. (Cancer Res., 66:2970-2979 (2006)) and overall survival in the 2019 cohort (Mansfield et al., J. Thorac. Oncol., 14 (2): 276-287 (2019)) are shown. FIG. 5B) The ROC curve based on logistic regression predicting NSB and SB from the 2019 cohort is shown.

FIG. 6. The number of junctions from the 2019 cohort and the current study are presented.

### DETAILED DESCRIPTION

This document provides methods and materials that can be used to determine whether or not a mammal (e.g., a human) having mesothelioma (e.g., mesothelioma with a high junction burden) is likely to respond a particular cancer treatment (e.g., immunotherapy with one or more immune checkpoint inhibitors). For example, a sample obtained from a mammal having mesothelioma with a high junction burden can be assessed for the presence or absence of an elevated level of polypeptides expressed by at least (e.g., 6, 8, 14, 17, 24, or more) APP genes in the sample to determine whether or not the mammal is likely to respond a particular cancer treatment (e.g., immunotherapy with one or more immune checkpoint inhibitors). In some cases, the methods and materials provided herein also can include administering one or more cancer treatments (e.g., one or more cancer treatments selected based, at least in part, on whether or not the mammal is likely to likely to respond a particular cancer treatment such as immunotherapy with one or more immune checkpoint inhibitors) to a mammal having mesothelioma to treat the mammal

A mammal (e.g., a human) having mesothelioma (e.g., mesothelioma with a high junction burden) can be assessed to determine whether or not the mammal is likely to respond a particular cancer treatment (e.g., immunotherapy with one or more immune checkpoint inhibitors) by detecting the presence or absence of an elevated level of at least 6 different APP polypeptides in a sample obtained from the mammal. As described herein, the presence of a high junction burden in combination with the presence or absence of an elevated level of at least 6 different APP in a sample obtained from a mammal having mesothelioma can be used to determine whether or not that mammal is likely to respond a particular cancer treatment (e.g., immunotherapy with one or more immune checkpoint inhibitors).

Any appropriate mammal having mesothelioma (e.g., mesothelioma with a high junction burden) can be assessed and/or treated as described herein. Examples of mammals that can have mesothelioma and can be assessed and/or treated as described herein include, without limitation, humans, non-human primates (e.g., monkeys), dogs, cats, horses, cows, pigs, sheep, mice, and rats. In some cases, a mammal having mesothelioma that can be assessed and/or treated as described herein can have been exposed to asbestos. In some cases, a mammal having mesothelioma that can be assessed and/or treated as described herein can have been administered therapeutic radiation. In some cases, a mammal having mesothelioma that can be assessed and/or treated as described herein can have one or more loss of function mutations in one or both copies of the mammal's BRCA1 Associated Protein 1 (BAP1) gene.

When assessing and/or treating a mammal (e.g., a human) having mesothelioma as described herein, the mesothelioma can be any type of mesothelioma. A mesothelioma can be any stage of mesothelioma (e.g., stage I, stage II, stage III, or stage IV). A mesothelioma can involve in any mesothelium tissue. For example, a mesothelioma can involve a tissue that surrounds the lungs (pleura), tissue that surrounds the abdomen (peritoneum), tissue that surrounds the heart (pericardium), and/or tissue that surrounds the testes (tunica vaginalis). A mesothelioma can include any cell type(s). For example, a mesothelioma can include epithelioid cells and/or sarcomatoid cells. In some cases, a mesothelioma can be biphasic (e.g., can include both epithelioid cells sarcomatoid cells). Examples of types of mesothelioma a mammal (e.g., a human) being assessed and/or treated as described herein can have include, without limitation, pleural mesothelioma (e.g., MPM), peritoneal mesothelioma, pericardial mesothelioma, and paratesticular mesothelioma. In some and cases, a mesothelioma can be a primary cancer (e.g., a localized primary cancer). In some cases, a mesothelioma can have metastasized. In some cases, a mesothelioma can be an unresectable mesothelioma. In some cases, a mesothelioma can be a CD8″ mesothelioma.

In some cases, the methods described herein can include identifying a mammal (e.g., a human) as having mesothelioma (e.g., mesothelioma with a high junction burden). Any appropriate method can be used to identify a mammal as having mesothelioma. For example, physical examination (e.g., to check for lumps), imaging techniques (e.g., X-rays such as such as a chest X-ray, computerized tomography (CT) scanning, magnetic resonance imaging (MRI), and positron emission tomography (PET)), laboratory examination (e.g., of a sample such as a tissue sample obtained by biopsy), and/or surgery can be used to identify a mammal (e.g., a human) as having mesothelioma.

In some cases, a mammal (e.g., a human) to be assessed and/or treated as described herein can have mesothelioma with a high junction burden. For example, a high junction burden in combination with an elevated level of at least 6 different APP polypeptides) can indicate that a mammal is likely to respond a particular cancer treatment (e.g., immunotherapy with one or more immune checkpoint inhibitors).

A high junction burden can include any number of junctions that is greater than 17. In some cases, a high junction burden can include at least 17 junctions per megabase (mb). In some cases, a high junction burden can include from about 17 junctions to about 1000 junctions (e.g., from about 17 junctions to about 800 junctions, from about 17 junctions to about 500 junctions, from about 17 junctions to about 200 junctions, from about 17 junctions to about 100 junctions, from about 17 junctions to about 70 junctions, from about 17 junctions to about 50 junctions, from about 17 junctions to about 30 junctions, from about 50 junctions to about 1000 junctions, from about 100 junctions to about 1000 junctions, from about 300 junctions to about 1000 junctions, from about 500 junctions to about 1000 junctions, from about 700 junctions to about 1000 junctions, from about 20 junctions to about 700 junctions, from about 30 junctions to about 500 junctions, from about 40 junctions to about 300 junctions, from about 50 junctions to about 200 junctions, from about 60 junctions to about 100 junctions, from about 20 junctions to about 50 junctions, from about 50 junctions to about 100 junctions, from about 100 junctions to about 200 junctions, from about 200 junctions to about 300 junctions, from about 300 junctions to about 400 junctions, from about 400 junctions to about 500 junctions, from about 500 junctions to about 600 junctions, from about 600 junctions to about 700 junctions, from about 700 junctions to about 800 junctions, or from about 800 junctions to about 900 junctions). For example, a high junction burden can include from about 23 junctions per mb to about 348 junctions per mb (e.g., about 130 junctions per mb).

A junction burden can include any type of junction. A junction can be at the break of any type of chromosomal rearrangement (e.g., insertions, deletions, translocations, and inversions). In some cases, a junction can include a single junction (e.g., a single junction present at a deletion, insertion, or translocation). In some cases, a junction can include two junctions (e.g., first junction present on one end of an inversion or a balanced translocation, and a second junction present on the other end of the inversion or the balanced translocation). In some cases, a junction can include multiple junctions (e.g., two or more junctions present where each junction is present at a break of a three-way, four-way, or more translocation). A junction can include one or more breaks in any appropriate genomic nucleic acid or gene. Examples of genes that can include one or more junctions within a genome having a high junction burden include, without limitation, ABL1, BOR, and CYP19.

In some cases, a mesothelioma can be identified as having a high junction burden. Any appropriate method can be used to identify the presence or absence of a high junction burden. For example, sequencing such as mate pair library sequencing, whole genome sequencing, and RNA sequencing can be used to identify the presence or absence of a high junction burden within mesothelioma cells. In some cases, the presence or absence of a high junction burden can be identified as described in Example 1.

In some cases, a mammal (e.g., a human) having mesothelioma (e.g., mesothelioma with a high junction burden) can be identified as being likely to respond a particular cancer treatment (e.g., immunotherapy with one or more immune checkpoint inhibitors) based, at least in part, on the presence of an elevated level of at least 6 different APP polypeptides in a sample obtained from the mammal. In some cases, an APP polypeptide can be involved in the regulation of antigen processing. In some cases, an APP polypeptide can be involved in the presentation of a peptide antigen. In some cases, an APP polypeptide can be involved in processing and/or presentation of an endogenous peptide antigen. In some cases, an APP polypeptide can be involved in processing and/or presentation via the MHC Class I ER pathway. In some cases, an APP polypeptide can be involved in processing and/or presentation via the MHC Class IB pathway. Examples of nucleic acids that can encode an APP polypeptide having an elevated level in a mammal identified as being likely to respond a particular cancer treatment (e.g., immunotherapy with one or more immune checkpoint inhibitors) as described herein include, without limitation, a PYCARD) gene, a HFE gene, a HLA-DOA gene, a HLA-DOB gene, a TREM2 gene, a TAPBPL gene, a HLA-A gene, a HLA-B gene, a HLA-C gene, a HLA-E gene, a HLA-F gene, a HLA-G gene, a HLA-H gene, a AZGP1 gene, a B2M gene, a TAP2 gene, a ABCB9 gene, a IDE gene, a ERAP1 gene, a ERAP2 gene, a TAP1 gene, a TAPBP gene, a AP3B1 gene, a AP3D1 gene, a CD1A gene, a CD1B gene, a CD1C gene, a CD1D gene, a CD1E gene, a ATG5 gene, and a CD74 gene.

Examples of APP polypeptides that can be assessed to determine if an elevated level is present or absent within a sample (e.g., a sample of mesothelioma cells) as described herein include, without limitation, a PYCARD polypeptide, a HFE polypeptide, a HLA-DOA polypeptide, a HLA-DOB polypeptide, a TREM2 polypeptide, a TAPBPL polypeptide, a HLA-A polypeptide, a HLA-B polypeptide, a HLA-C polypeptide, a HLA-E polypeptide, a HLA-F polypeptide, a HLA-G polypeptide, a HLA-H polypeptide, a AZGP1 polypeptide, a B2M polypeptide, a TAP2 polypeptide, a ABCB9 polypeptide, a IDE polypeptide, a ERAP1 polypeptide, a ERAP2 polypeptide, a TAP1 polypeptide, a TAPBP polypeptide, a AP3B1 polypeptide, a AP3D1 polypeptide, a CD1A polypeptide, a CD1B polypeptide, a CD1C polypeptide, a CD1D polypeptide, a CD1E polypeptide, a ATG5 polypeptide, and a CD74 polypeptide. In some cases, an APP polypeptide can be encoded by an APP nucleic acid or gene listed in Table 4. The term “elevated level” as used herein with respect to an APP polypeptide in a sample refers to any level that is higher than a reference level of the APP polypeptide. The term “reference level” as used herein with respect to a level of APP polypeptide refers to the level of the APP polypeptide typically observed in a control sample. Control samples can include, without limitation, samples from one or more healthy mammals (e.g., healthy humans), and one or more non-cancerous cells lines. In some cases, an elevated level of an APP polypeptide can be a level that is at least 2 (e.g., at least 5, at least 10, at least 15, at least 20, at least 25, at least 35, or at least 50) fold greater than a reference level of the APP polypeptide. It will be appreciated that levels of polypeptide expression from comparable samples are used when determining whether or not a particular level is an elevated level of polypeptide expression. For example, when assessing a human sample for an elevated level of a human PYCARD polypeptide, then the reference level of PYCARD polypeptide expression can be that observed in healthy human cells.

In some cases, a mammal (e.g., a human) having mesothelioma (e.g., mesothelioma with a high junction burden) can be identified as being likely to respond a particular cancer treatment (e.g., immunotherapy with one or more immune checkpoint inhibitors) based, at least in part, on the presence of an elevated level of at least 6 different APP polypeptides in a sample obtained from the mammal). For example, a mammal having mesothelioma with a high junction burden can be identified as being likely to respond a particular cancer treatment (e.g., immunotherapy with one or more immune checkpoint inhibitors) based, at least in part, on the presence of an elevated level for at least 6 different APP polypeptides (e.g., from about 6 APP polypeptides to about 31 APP polypeptides (e.g., from about 6 APP polypeptides to about 28 APP polypeptides, from about 6 APP polypeptides to about 25 APP polypeptides, from about 6 APP polypeptides to about 22 APP polypeptides, from about 6 APP polypeptides to about 20 APP polypeptides, from about 6 APP polypeptides to about 17 APP polypeptides, from about 6 APP polypeptides to about 15 APP polypeptides, from about 6 APP polypeptides to about 12 APP polypeptides, from about 6 APP polypeptides to about 10 APP polypeptides, from about 8 APP polypeptides to about 31 APP polypeptides, from about 10 APP polypeptides to about 31 APP polypeptides, from about 13 APP polypeptides to about 31 APP polypeptides, from about 15 APP polypeptides to about 31 APP polypeptides, from about 17 APP polypeptides to about 31 APP polypeptides, from about 20 APP polypeptides to about 31 APP polypeptides, from about 24 APP polypeptides to about 31 APP polypeptides, from about 8 APP polypeptides to about 24 APP polypeptides, from about 10 APP polypeptides to about 20 APP polypeptides, from about 8 APP polypeptides to about 12 APP polypeptides, from about 12 APP polypeptides to about 18 APP polypeptides, or from about 18 APP polypeptides to about 24 APP polypeptides)).

In some cases, a mammal (e.g., a human) having mesothelioma (e.g., mesothelioma with a high junction burden) can be identified as being likely to respond a particular cancer treatment (e.g., immunotherapy with one or more immune checkpoint inhibitors) based, at least in part, on the presence of an elevated level of at least a PYCARD polypeptide, a HFE polypeptide, a HLA-DOA polypeptide, a HLA-DOB polypeptide, a TREM2 polypeptide, and a TAPBPL polypeptide in a sample obtained from the mammal.

In some cases, a mammal (e.g., a human) having mesothelioma (e.g., mesothelioma with a high junction burden) can be identified as being likely to respond a particular cancer treatment (e.g., immunotherapy with one or more immune checkpoint inhibitors) based, at least in part, on the presence of an elevated level of at least a HLA-A polypeptide, a HLA-B polypeptide, a HLA-C polypeptide, a HLA-E polypeptide, a HLA-F polypeptide, a HLA-G polypeptide, a HLA-H polypeptide, and a AZGP1 polypeptide in a sample obtained from the mammal.

In some cases, a mammal (e.g., a human) having mesothelioma (e.g., mesothelioma with a high junction burden) can be identified as being likely to respond a particular cancer treatment (e.g., immunotherapy with one or more immune checkpoint inhibitors) based, at least in part, on the presence of an elevated level of at least a HLA-E polypeptide, a HLA-F polypeptide, a HLA-G polypeptide, a HLA-H polypeptide, a AZGP1 polypeptide, a B2M polypeptide, and a TAP2 polypeptide in a sample obtained from the mammal.

In some cases, a mammal (e.g., a human) having mesothelioma (e.g., mesothelioma with a high junction burden) can be identified as being likely to respond a particular cancer treatment (e.g., immunotherapy with one or more immune checkpoint inhibitors) based, at least in part, on the presence of an elevated level of at least a ABCB9 polypeptide, a HFE polypeptide, a HLA-A polypeptide, a HLA-B polypeptide, a HLA-C polypeptide, a HLA-E polypeptide, a HLA-F polypeptide, a HLA-G polypeptide, a HLA-H polypeptide, a IDE polypeptide, a ERAP1 polypeptide, a AZGP1 polypeptide, a B2M polypeptide, a ERAP2 polypeptide, a TAP1 polypeptide, a TAP2 polypeptide, and a TAPBP polypeptide in a sample obtained from the mammal.

In some cases, a mammal (e.g., a human) having mesothelioma (e.g., mesothelioma with a high junction burden) can be identified as being likely to respond a particular cancer treatment (e.g., immunotherapy with one or more immune checkpoint inhibitors) based, at least in part, on the presence of an elevated level of at least a HLA-E polypeptide, a HLA-F polypeptide, a HLA-G polypeptide, a HLA-H polypeptide, a AZGP1 polypeptide, a B2M polypeptide, a TAP2 polypeptide, a AP3B1 polypeptide, a AP3D1 polypeptide, a CD1A polypeptide, a CD1B polypeptide, a CD1C polypeptide, a CD1D polypeptide, and a CD1E polypeptide in a sample obtained from the mammal.

In some cases, a mammal (e.g., a human) having mesothelioma (e.g., mesothelioma with a high junction burden) can be identified as being likely to respond a particular cancer treatment (e.g., immunotherapy with one or more immune checkpoint inhibitors) based, at least in part, on the presence of an elevated level of at least a ABCB9 polypeptide, a HFE polypeptide, a HLA-A polypeptide, a HLA-B polypeptide, a HLA-C polypeptide, a HLA-E polypeptide, a HLA-F polypeptide, a HLA-G polypeptide, a HLA-H polypeptide, a IDE polypeptide, a ERAP1 polypeptide, a AZGP1 polypeptide, a B2M polypeptide, a ERAP2 polypeptide, a TAP1 polypeptide, a TAP2 polypeptide, a TAPBP polypeptide, a CD1A polypeptide, a CD1B polypeptide, a CD1C polypeptide, a CD1D polypeptide, a CD1E polypeptide, a ATG5 polypeptide, and a CD74 polypeptide in a sample obtained from the mammal.

Any appropriate method can be used to identify the presence, absence, or level of an APP polypeptide. In some cases, the presence, absence, or level of an APP polypeptide can be assessed by detecting and/or quantifying mRNA encoding an APP polypeptide. Examples of methods that can be used to detect and/or quantify mRNA include, without limitation, RT-PCR techniques (e.g., quantitative RT-PCR techniques), and RNA sequencing. In some cases, the presence, absence, or level of an APP polypeptide can be assessed by detecting and/or quantifying the APP polypeptide. Examples of methods that can be used to detect and/or quantify APP polypeptides include, without limitation, immunohistochemistry (IHC) techniques, mass spectrometry techniques (e.g., proteomics-based mass spectrometry assays or targeted quantification-based mass spectrometry assays), western blotting techniques, and enzyme-linked immunosorbent assays (ELISAs). In some cases, the presence, absence, or level of an APP polypeptide can be identified as described in Example 1.

Any appropriate sample from a mammal (e.g., a human) having mesothelioma (e.g., mesothelioma with a high junction burden) can be assessed as described herein (e.g., to determine whether or not the mammal is likely to respond a particular cancer treatment such as immunotherapy with one or more immune checkpoint inhibitors based, at least in part, on the presence or absence of an elevated level of at least 6 different APP polypeptides in a sample obtained from the mammal). In some cases, a sample can be a biological sample. In some cases, a sample can contain one or more cancer cells (e.g., mesothelioma cells). In some cases, a sample can contain one or more biological molecules (e.g., nucleic acids such as DNA and RNA, polypeptides, carbohydrates, lipids, hormones, and/or metabolites). For example, a sample can contain genomic DNA. Examples of samples that can be assessed as described herein include, without limitation, fluid samples (e.g., whole blood, serum, plasma, urine, and saliva), tissue samples (e.g., mesothelium tissue such as pleural, peritoneal, pericardial, and tunica vaginalis tissues), cellular samples (e.g., buccal swabs), mesothelioma samples, and mesothelium samples. A sample can be a fresh sample or a fixed sample (e.g., a formaldehyde-fixed sample or a formalin-fixed sample). In some cases, one or more biological molecules can be isolated from a sample. For example, nucleic acid can be isolated from a sample and can be assessed as described herein. In some cases, polypeptides can be isolated from a sample and can be assessed as described herein.

In some cases, a mammal (e.g., a human) having mesothelioma (e.g., mesothelioma with a high junction burden) and identified as being likely to respond a particular cancer treatment (e.g., immunotherapy with one or more immune checkpoint inhibitors) as described herein (e.g., based, at least in part, on the presence of an elevated level of at least 6 different APP polypeptides) can be selected for treatment with one or more immunotherapies (e.g., one or more immune checkpoint inhibitors). For example, a mammal having mesothelioma with a high junction burden and identified as having the presence of an elevated level of at least 6 different APP polypeptides in a sample obtained from the mammal can be selected to receive one or more (e.g., one, two, three, four, five, or more) immunotherapies.

In some cases, a mammal (e.g., a human) having mesothelioma (e.g., mesothelioma with a high junction burden) and identified as not being likely to respond a particular cancer treatment (e.g., immunotherapy with one or more immune checkpoint inhibitors) as described herein (e.g., based, at least in part, on the absence of an elevated level of at least 6 different APP polypeptides) can be selected for treatment with one or more alternative cancer treatments (e.g., one or more cancer treatments that are not an immunotherapy). For example, a mammal having mesothelioma with a high junction burden and identified as lacking an elevated level of at least 6 different APP polypeptides in a sample obtained from the mammal can be selected to receive one or more (e.g., one, two, three, four, five, or more) alternative cancer treatments.

This document also provides methods for treating a mammal (e.g., a human) having mesothelioma (e.g., mesothelioma with a high junction burden). In some cases, a mammal (e.g., a human) having mesothelioma with a high junction burden and assessed as described herein (e.g., to determine whether or not the mammal is likely to respond a particular cancer treatment such as immunotherapy with one or more immune checkpoint inhibitors based, at least in part, on the presence or absence of an elevated level of at least 6 different APP polypeptides in a sample obtained from the mammal) can be administered or instructed to self-administer one or more (e.g., one, two, three, four, five, or more) cancer treatments, where the one or more cancer treatments are effective to treat the cancer within the mammal. For example, a mammal having mesothelioma with a high junction burden can be administered or instructed to self-administer one or more cancer treatments selected based, at least in part, on whether or not the mammal is likely to respond a particular cancer treatment such as immunotherapy with one or more immune checkpoint inhibitors (e.g., based, at least in part, on the presence or absence of an elevated level of at least 6 different APP polypeptides in a sample obtained from the mammal).

In general, a cancer treatment for mesothelioma (e.g., mesothelioma with a high junction burden) can include any appropriate mesothelioma cancer treatment. In some cases, a cancer treatment can include administering one or more cancer drugs (e.g., chemotherapeutic agents, targeted cancer drugs, immunotherapy drugs, corticosteroids, and hormones) to a mammal in need thereof. Examples of cancer drugs that can be administered to a mammal having mesothelioma (e.g., mesothelioma with a high junction burden) can include, without limitation, pembrolizumab (e.g., KEYTRUDA®), nivolumab (e.g., OPDIVO®), cemiplimab (e.g., LIBTAYO®), atezolizumab (e.g., TECENTRIQ®), avelumab (e.g., Bavencio®), durvalumab (e.g., IMFINZI®), ipilimumab (e.g., YERVOY®), cisplatin (e.g., PLATINOL®), pemetrexed (e.g., Alimta® and PEMFEXY™), carboplain, bevacizumab, vinorelbine, gemcitabine, ramucirumab, and combinations thereof. In some cases, a cancer treatment for mesothelioma (e.g., mesothelioma with a high junction burden) can include surgery and other medical interventions. Examples of surgeries and other medical interventions that can be performed on a mammal having mesothelioma (e.g., mesothelioma with a high junction burden) to treat the mammal include, without limitation, surgery (e.g., to decrease fluid buildup in the chest (pleurodesis), to remove the tissue around the lungs (pleurectomy), to remove a lung and the surrounding tissue, and for peritoneal mesothelioma), radiation therapy, tumor treatment fields, and cryotherapy.

When treating a mammal (e.g., a human) having mesothelioma (e.g., mesothelioma with a high junction burden) and identified as being likely to respond a particular cancer treatment (e.g., immunotherapy with one or more immune checkpoint inhibitors) as described herein (e.g., based, at least in part, on the presence of an elevated level of at least 6 different APP polypeptides), the mammal can be administered or instructed to self-administer one or more (e.g., one, two, three, four, five, or more) immunotherapies (e.g., one or more immune checkpoint inhibitors). For example, a mammal having mesothelioma with a high junction burden and identified as having the presence of an elevated level of at least 6 different APP polypeptides in a sample obtained from the mammal can be administered or instructed to self-administer one or more immune checkpoint inhibitors. In some cases, an immune checkpoint inhibitor can inhibit (e.g., can reduce) PD-1 signaling (e.g., can inhibit polypeptide expression or polypeptide activity of a programmed cell death protein 1 (PD-1) receptor polypeptide or can inhibit polypeptide expression or polypeptide activity of a programmed death-ligand 1 (PD-L1) polypeptide). In some cases, an immune checkpoint inhibitor can inhibit (e.g., can reduce) cytotoxic T-lymphocyte-associated protein 4 (CTLA-4) signaling (e.g., can inhibit polypeptide expression or polypeptide activity of a CTLA-4 polypeptide). Examples of immune checkpoint inhibitors that can be administered to a mammal (e.g., a human) having mesothelioma with a high junction burden and having the presence of an elevated level of at least 6 different APP polypeptides in a sample obtained from the mammal to treat the mammal include, without limitation, pembrolizumab (e.g., KEYTRUDA®), nivolumab (e.g., OPDIVO®), cemiplimab (e.g., LIBTAYO®), atezolizumab (e.g., TECENTRIQ®), avelumab (e.g., Bavencio®), durvalumab (e.g., IMFINZI®), and ipilimumab (e.g., YERVOY®).

In some cases, when treating a mammal (e.g., a human) having mesothelioma (e.g., mesothelioma with a high junction burden) and identified as being likely to respond a particular cancer treatment (e.g., immunotherapy with one or more immune checkpoint inhibitors) as described herein (e.g., based, at least in part, on the presence of an elevated level of at least 6 different APP polypeptides) the one or more (e.g., one, two, three, four, five, or more) immunotherapies (e.g., one or more immune checkpoint inhibitors) can be the sole active agent(s) administered to the mammal to treat the mesothelioma.

In some cases, when treating a mammal (e.g., a human) having mesothelioma (e.g., mesothelioma with a high junction burden) and identified as being likely to respond a particular cancer treatment (e.g., immunotherapy with one or more immune checkpoint inhibitors) as described herein (e.g., based, at least in part, on the presence of an elevated level of at least 6 different APP polypeptides) the one or more (e.g., one, two, three, four, five, or more) immunotherapies (e.g., one or more immune checkpoint inhibitors) can be administered to the mammal together with one or more additional agents/therapies used to treat mesothelioma. Examples of anti-cancer treatments that can be administered to a mammal (e.g., a human) having mesothelioma together with one or more immunotherapies (e.g., one or more immune checkpoint inhibitors) include, without limitation, administering one or more chemotherapy drugs such as cisplatin (e.g., PLATINOL®), pemetrexed (e.g., Alimta® and PEMFEXY™), carboplain, bevacizumab, vinorelbine, gemcitabine, ramucirumab, and any combinations thereof. In cases where one or more immunotherapies (e.g., one or more immune checkpoint inhibitors) are used in combination with additional agents used to treat cancer, the one or more additional agents can be administered at the same time (e.g., in a single composition containing both one or more immunotherapies and the one or more additional agents) or independently. For example, one or more immunotherapies (e.g., one or more immune checkpoint inhibitors) can be administered first, and the one or more additional agents administered second, or vice versa. Examples of therapies that can be used to treat cancer include, without limitation, surgery (e.g., to decrease fluid buildup in the chest (pleurodesis), to remove the tissue around the lungs (pleurectomy), to remove a lung and the surrounding tissue, and for peritoneal mesothelioma), radiation therapy, tumor treatment fields, and cryotherapy. In cases where one or more immunotherapies (e.g., one or more immune checkpoint inhibitors) are used in combination with one or more additional therapies used to treat mesothelioma, the one or more additional therapies can be performed at the same time or independently of the administration of the one or more immunotherapies. For example, one or more immunotherapies (e.g., one or more immune checkpoint inhibitors) can be administered before, during, or after the one or more additional therapies are performed.

When treating a mammal (e.g., a human) having mesothelioma (e.g., mesothelioma with a high junction burden) and identified as not being likely to respond a particular cancer treatment (e.g., immunotherapy with one or more immune checkpoint inhibitors) as described herein (e.g., based, at least in part, on the absence of an elevated level of at least 6 different APP polypeptides), the mammal can be administered or instructed to self-administer one or more alternative cancer treatments (e.g., one or more cancer treatments that are not an immunotherapy). For example, a mammal having mesothelioma with a high junction burden and identified as lacking the presence of an elevated level of at least 6 different APP polypeptides in a sample obtained from the mammal can be administered or instructed to self-administer one or more alternative cancer treatments. Examples of alternative cancer treatments that can be administered to a mammal (e.g., a human) having mesothelioma with a high junction burden and lacking the presence of an elevated level of at least 6 different APP polypeptides in a sample obtained from the mammal to treat the mammal include, without limitation, administering one or more chemotherapy drugs (e.g., cisplatin (e.g., PLATINOL®), pemetrexed (e.g., Alimta® and PEMFEXY™), carboplain, bevacizumab, vinorelbine, gemcitabine, and ramucirumab), surgery (e.g., to decrease fluid buildup in the chest (pleurodesis), to remove the tissue around the lungs (pleurectomy such as pleurectomy and decortication, extended pleurectomy and decortication, and extrapleural pneumonectomy), to remove a lung and the surrounding tissue, and for peritoneal mesothelioma), radiation therapy, tumor treatment fields, and. cryotherapy.

In some cases, a mammal (e.g., a human) having mesothelioma (e.g., mesothelioma with a high junction burden) and identified as not being likely to respond a particular cancer treatment (e.g., immunotherapy with one or more immune checkpoint inhibitors) as described herein (e.g., based, at least in part, on the absence of an elevated level of at least 6 different APP polypeptides) is not administered an immunotherapy (e.g., an immune checkpoint inhibitor).

In some cases, when treating a mammal (e.g., a human) having mesothelioma (e.g., mesothelioma with a high junction burden) as described herein, the treatment can be effective to treat the mesothelioma. For example, the number of cancer cells present within a mammal can be reduced using the methods and materials described herein. In some cases, the methods and materials described herein can be used to reduce the number of cancer cells present within a mammal having mesothelioma by, for example, 10, 20, 30, 40, 50, 60, 70, 80, 90, 95, or more percent. In some cases, the number of cancer cells present within a mammal does not increase. For example, the size (e.g., volume) of one or more tumors present within a mammal can be reduced using the methods and materials described herein. In some cases, the methods and materials described herein can be used to reduce the size of one or more tumors present within a mammal having mesothelioma by, for example, 10, 20, 30, 40, 50, 60, 70, 80, 90, 95, or more percent. In some cases, the size (e.g., volume) of one or more tumors present within a mammal does not increase.

In some cases, when treating a mammal (e.g., a human) having mesothelioma (e.g., mesothelioma with a high junction burden) as described herein, the treatment can be effective to improve survival of the mammal. For example, the methods and materials described herein can be used to improve disease-free survival (e.g., relapse-free survival). For example, the methods and materials described herein can be used to improve overall survival. For example, the methods and materials described herein can be used to improve the survival of a mammal having mesothelioma by, for example, 10, 20, 30, 40, 50, 60, 70, 80, 90, 95, or more percent. For example, the methods and materials described herein can be used to improve the survival of a mammal having mesothelioma by, for example, at least 6 months (e.g., about 6 months, about 8 months, about 10 months, about 1 year, about 1.5 years, about 2 years, about 2.5 years, or about 3 years).

In some cases, when treating a mammal (e.g., a human) having mesothelioma (e.g., mesothelioma with a high junction burden) as described herein, the treatment can be effective to reduce or eliminate one or more symptoms of the mesothelioma. Examples of symptoms mesothelioma that can be reduced or eliminated using the methods and materials described herein can include, without limitation, chest pain, painful coughing, shortness of breath, unusual lumps of tissue under the skin on your chest, unexplained weight loss, abdominal pain, abdominal swelling, nausea, unexplained weight loss, difficulty breathing, and chest pains. For example, the methods and materials described herein can be used to reduce one or more symptoms of mesothelioma within a mammal having mesothelioma by, for example, 10, 20, 30, 40, 50, 60, 70, 80, 90, 95, or more percent.

In some cases, when treating a mammal (e.g., a human) having mesothelioma (e.g., mesothelioma with a high junction burden) as described herein, the treatment can be effective to reduce or eliminate one or more complications associated with the mesothelioma. Examples of complications of mesothelioma that can be reduced or eliminated using the methods and materials described herein include, without limitation, difficulty breathing, chest pain, difficulty swallowing, pain (e.g., caused by pressure on the nerves and spinal cord), and accumulation of fluid in the chest (pleural effusion). For example, the methods and materials described herein can be used to reduce one or more complications associated with mesothelioma within a mammal having mesothelioma by, for example, 10, 20, 30, 40, 50, 60, 70, 80, 90, 95, or more percent.

In some cases, a course of treatment, the number of cancer cells present within a mammal, and/or the severity of one or more symptoms related to the mesothelioma (e.g., mesothelioma with a high junction burden) can be monitored. Any appropriate method can be used to determine whether or not the number of cancer cells present within a mammal is reduced. For example, imaging techniques can be used to assess the number of cancer cells present within a mammal.

The invention will be further described in the following examples, which do not limit the scope of the invention.

### EXAMPLES

### Example 1: Chromosomal Rearrangements and Antigen Presentation as Predictors of Survival in Mesothelioma Treated with Immune Checkpoint Inhibitors

This Example describes the analysis of structural variants and antigen presentation gene set expression may facilitate patient selection for immune checkpoint inhibitors.

### Methods

### Patients and Specimens

Biopsies were obtained from patients just prior to treatment with nivolumab (NCT02497508) or nivolumab with ipilimumab (NCT03048474), after previous treatment with platinum-based chemotherapy. DNA and RNA were purified using the AllPrep DNA/RNA/miRNA Universal kit (Qiagen, #80224) following the instructions provided by the manufacturer. The buffer included B-mercaptoethanol for the specimens obtained from NCT02497508, and dithiothreitol for the ones obtained from NCT03048474. Otherwise, there were no differences in the handling of the specimens or nucleic acid purification. The clinical trials and translational studies were approved by the local institutional ethics committees. Characteristics of the patients included in the analysis were compared to those of patients who were excluded due to insufficient materials using the Fisher's exact test for categorical variables and the Mann Whitney U test for continuous variables. Survival between these groups was compared using the R packages “survival” and “survminer.”

### Determination of Junction Burdens

Chromosomal rearrangements were reported by sequencing DNA prepared according to the mate-pair whole-genome library protocol (Nextera Library Prep Protocol). Sequencing results were mapped by BIMA, and the junctions of the chromosomal rearrangements were called by SVAtools. BIMA and SVAtools are Mayo Clinic in-house informatic pipelines. The junctions of the chromosomal rearrangements were annotated with 1) the position of the junction with a resolution of 200-500 bp, 2) direction of the chromosomal rearrangement and 3) genes at the junction using NCBI RefSeq genes for GRCh38. The number of chromosomal rearrangements per sample was assessed by counting the number of unique genes hit by all junctions in the sample. All specimens had 60× or greater bridged coverage for the detection of junctions, except one, which had 40× bridged coverage. Chromosomal rearrangements may refer to insertions, deletions, translocations, and inversions. Junctions are the locations of the breaks of these chromosomal rearrangements. There may be one junction (e.g., for a deletion, insertion, or translocation), two junctions (e.g., for an inversion or a balanced translocation), or multiple junctions (e.g., for a three-way, a four-way, or more complex translocation) involved with each chromosomal rearrangement.

### RNA-Seq Analyses

Mapping of the RNA-seq data and estimations of gene expression counts in each sample were performed by MAP-RSeq pipeline. Raw “count” files were processed by the “edgeR” package to generate log 2 normalized gene expression values.

### Antigen Processing and Presentation

The biological processes gene ontology dataset in the molecular signature database was searched for gene-sets with names that included “antigen” and “presentation.” Of the 21 found hits, nine were eliminated for processes involving lipid, polysaccharide, or exogenous antigens or for processes representing dendritic cell or T-cell APP. Single sample enrichment scores in the remaining 12 gene-sets were calculated by using the “ssGSEA” algorithm in the “GSVA” package.

### Survival and Immune Checkpoint Inhibitor Survival Analyses

Associations of interactions between gene-sets and log 2 transformed junction burden (APP*log 2 [junction burden]) with overall survival (OS) were found by using the “coxph” (cox proportional hazard) program in the “survival” package. Associations of these interactions with response to immune checkpoint inhibitors in terms of survivals at 1.5-year (S_{1.5 yr}) were calculated by logistic regression (LR) using the “glm” (generalized linear model) package. APP and junction burden interactions were considered significant when either or both of the following conditions were met: (i) log-rank p-values and the interaction terms in the OS models were significant (p<0.05), or (ii) the interaction terms in LR analysis was significant and the LR model had an accuracy based on area under the curve (AUC) greater than 0.7. To create the Kaplan Meir (KM) plot representing an individual gene set interaction with junction burden, samples were categorized as either “High” or “Low” by using the median multiplication product of gene-set scores and log 2 [junction burden] as the threshold. Reported p-values in the plot are associations of the interaction and the model (log rank test) with overall survival by “coxph” program.

### Forest Plots

Median enrichment scores in each of the APP gene-sets were used to group samples in high and low APP categories. In each category, hazard ratios representing associations between junction burdens and overall survival were calculated by “coxph” and plotted using the “forestplot” package.

### Examination of Existing Models

Immunotherapy response models described elsewhere (Fu et al., Genome Med., 12:21 (2020)) were examined for predicting significant benefit (SB) and no significant benefit (NSB). Log 2 transformed gene expression data were normalized in each row by subtracting average values across all samples according to the authors instructions. Normalized expression values were input to the python program “tidepy” to estimate individual tumor scores in 14 models. Logistic regression analyses were then used to estimate the accuracy of models with the CD8 model having been found as the best performer. Finally, “PROC” program was used to plot the ROC curves for TIDE, IFNG, PD-L1, and CD8 models.

### Results

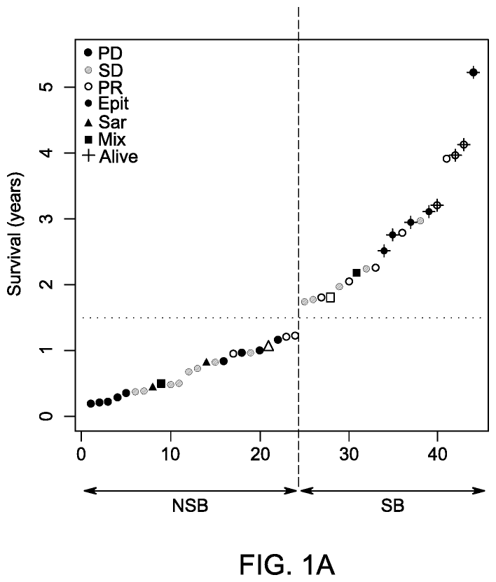

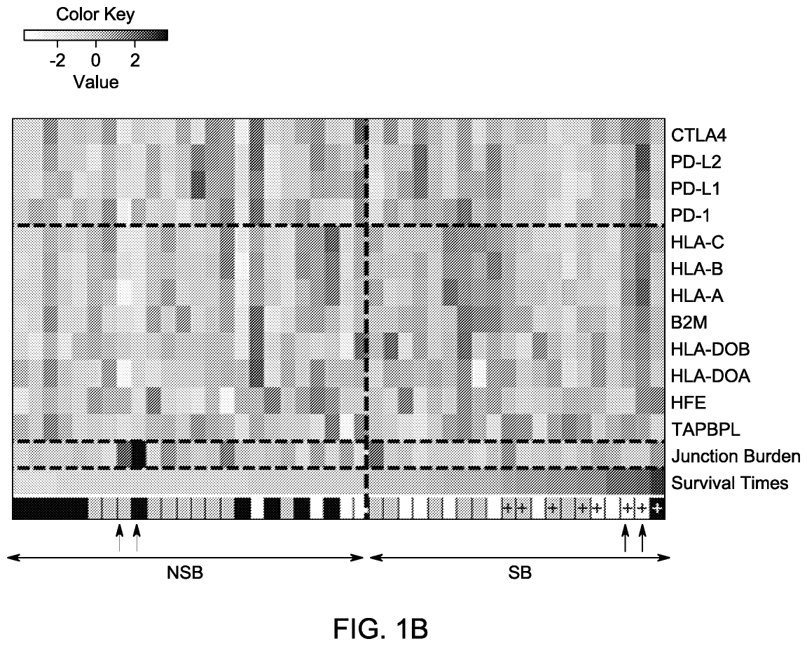

Sixty-eight patients with pleural mesothelioma were treated with the PD-1 inhibitor nivolumab alone or in combination with the CTLA-4 inhibitor ipilimumab on the NivoMes (n=34) and INITIATE (n=34) clinical trials, respectively (Quispel-Janssen et al., J. Thorac. Oncol.; 13:1569-1576 (2018); and Disselhorst et al., Lancet Respir. Med., 7:260-270 (2019)) (Table 1). These patients had received at least one prior line of platinum-containing therapy. Biopsies were obtained on 65 of these patients just prior to the start of treatment with an immune checkpoint inhibitor(s), and 44 of these specimens had sufficient DNA and RNA content for analysis. There were no significant differences between the characteristics of the patients included in this analysis and those excluded based on sample insufficiency including sex, trial treatment, performance status, line of therapy, age, or overall survival. Despite the historic median survivals of less than six months with second or later line therapy in mesothelioma, there was a separation in overall survival at 1.5 years (S_{1.5 yr}) from start of treatment on trial which we selected to group patients into categories of significant benefit (SB, >S_{1.5 yr}) and no significant benefit (NSB, ≤S_{1.5 yr}) (FIG. 1A). The biopsies obtained just prior to treatment were analyzed by mate-pair DNA sequencing and RNA-seq. There were many chromosomal rearrangements in each specimen (median 130 junctions, range 23-348), and a fraction of these involved unique genes (median 18, range 1-68). The chromosomal rearrangements involving unique genes in each tumor were selected for analysis given their potential to be expressed and refer to them as junction burdens from hereon.

TABLE 1
Patient characteristics.

| | Patients |
|---|---|
| Characteristics | (n = 44) |
| Sex, n (%) | |
| Male | 37 (84%) |
| Female | 7 (16%) |
| Histology, n (%) | |
| Epithelioid | 38 (86%) |
| Biphasic | 3 (7%) |
| Sarcomatoid | 3 (7%) |
| Treatment, n (%) | |
| Nivolumab | 24 (55%) |
| Nivolumab, Ipilimumab | 20 (45%) |
| Line of therapy, n (%) | |
| 2 | 38 (86%) |
| ≥3 | 6 (14%) |
| Age, median (range) | 66 (47-81) |

Given the findings of the neoantigenic potential of chromosomal rearrangements, it was sought to determine whether junction burdens were associated with survival in patients with mesothelioma treated with immune checkpoint inhibitors. No association was found between junction burdens and overall survival (Cox model log rank p>0.5). Two patients with the highest junction burdens had very short survival times, whereas two other patients with moderate junction burdens had a durable survival benefit (FIG. 1B). The two patients with the highest junction burdens and poor survival had low expression of genes involved in antigen processing and presentation (APP). On the other hand, patients with moderate junction burdens and more durable survival had very robust expression of APP associated genes.

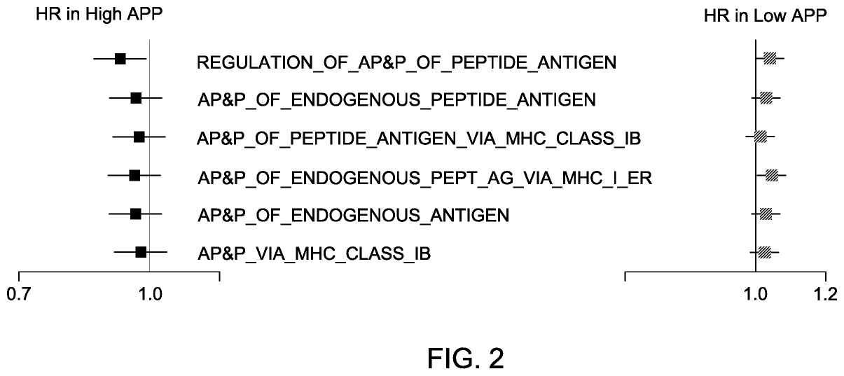

Since the impact of the junction burden appeared to be modulated by APP, it was examined whether the neoantigenic potential of chromosomal rearrangements was dependent upon the capability of cancer cells to present neo-antigens to the immune system. To examine whether there was an interaction between APP gene sets and junction burdens that impacted outcomes, 12 APP gene sets were selected from the Gene Ontology-Biological Processes dataset in the Molecular Signature Database and their enrichment scores were calculated. These scores were then used to test for interactions between APP gene sets and junction burdens on survival and found significant interactions with six APP gene sets (Table 2). Genes within each of these six APP gene sets are shown in Table 4. With these six APP gene sets, the hazard ratios representing associations between junction burdens and overall survival favored patients with high APP scores (all hazard ratios <1) more so than patients with low APP scores (all hazard ratios >1) (FIG. 2).

TABLE 2
Antigen processing and presentation gene set analysis.

| | P-IA- | p-Log | P-IA- | |
|---|---|---|---|---|
| | cox | Rank | lr | AUC |
| REGULATION_{—}OF_{—}AP&P_{—} | 0.0026 | 0.0031 | 0.0221 | 0.831 |
| OF_{—}PEPTIDE_{—}ANTIGEN | | | | |
| AP&P_{—}OF_{—}ENDOGENOUS_{—} | 0.021 | 0.041 | 0.040 | 0.724 |
| PEPTIDE_{—}ANTIGEN | | | | |
| AP&P_{—}OF_{—}PEPTIDE_{—} | 0.048 | 0.019 | 0.071 | 0.759 |
| ANTIGEN_{—}VIA_{—}MHC_{—} | | | | |
| CLASS_{—}IB | | | | |
| AP&P_{—}OF_{—}ENDOGENOUS_{—} | 0.049 | 0.010 | 0.072 | 0.811 |
| PEPTIDE_{—}ANTIGEN_{—}VIA_{—} | | | | |
| MHC_{—}CLASS_{—}I_{—}VIA_{—}ER_{—} | | | | |
| PATHWAY | | | | |
| AP&P_{—}OF_{—}ENDOGENOUS_{—} | 0.061 | 0.079 | 0.025 | 0.748 |
| ANTIGEN | | | | |
| AP&P_{—}VIA_{—}MHC_{—}CLASS_{—}IB | 0.154 | 0.045 | 0.023 | 0.800 |
| NEGATIVE_REGULATION_{—} | 0.034 | 0.145 | 0.072 | 0.702 |
| OF_AP&P | | | | |
| REGULATION_OF_AP&P | 0.065 | 0.253 | 0.079 | 0.697 |
| AP&P_OF_PEPTIDE_{—} | 0.080 | 0.318 | 0.240 | 0.610 |
| ANTIGEN_VIA_MHC_{—} | | | | |
| CLASS_I | | | | |
| POSITIVE_REGULATION_{—} | 0.197 | 0.382 | 0.100 | 0.660 |
| OF_AP&P | | | | |
| AP&P | 0.201 | 0.597 | 0.289 | 0.542 |
| AP&P_OF_PEPTIDE_{—} | 0.242 | 0.673 | 0.519 | 0.559 |
| ANTIGEN | | | | |

The statistical significance of interactions (P-IA-cox) and log-rank (p-Log Rank) in cox models, interactions (P-IA-lr) and area under the curve (AUC) in logistic regression models are listed. The gene sets with significant interactions are in bold. APP and junction burden interactions were considered significant when either or both of the following conditions were met: (i) log-rank p-values and the interaction terms in the OS models were significant (p < 0.05), or (ii) the interaction terms in LR analysis was significant and the LR model had an accuracy based on area under the curve (AUC) greater than 0.7.

TABLE 4
Genes in analysed gene sets.

REGULATION_OF_AP&P_OF_PEPTIDE_ANTIGEN: PYCARD, HFE, HLA-DOA, HLA-DOB, TREM2, TAPBPL
AP&P_OF_ENDOGENOUS_PEPTIDE_ANTIGEN: ABCB9, HFE, HLA-A, HLA-B, HLA-C, HLA-E, HLA-F, HLA-G, HLA-H, IDE, ERAP1,
AZGP1, B2M, ERAP2, TAP1, TAP2, TAPBP
AP&P_OF_PEPTIDE_ANTIGEN_VIA_MHC_CLASS_IB: HLA-E, HLA-F, HLA-G, HLA-H, AZGP1, B2M, TAP2, AP3B1, AP3D1, CD1A,
CD1B, CD1C, CD1D, CD1E
AP&P_OF_ENDOGENOUS_PEPTIDE_ANTIGEN_VIA_MHC_CLASS_I_VIA_ER_PATHWAY: HLA-A, HLA-B, HLA-C, HLA-E, HLA-F,
HLA-G, HLA-H, AZGP1
AP&P_OF_ENDOGENOUS_ANTIGEN: ABCB9, HFE, HLA-A, HLA-B, HLA-C, HLA-E, HLA-F, HLA-G, HLA-H, IDE, ERAP1, AZGP1,
B2M, ERAP2, TAP1, TAP2, TAPBP, CD1A, CD1B, CD1C, CD1D, CD1E, ATG5, CD74
AP&P_VIA_MHC_CLASS_IB: HLA-E, HLA-F, HLA-G, HLA-H, AZGP1, B2M, TAP2, AP3B1, AP3D1, CD1A, CD1B, CD1C, CD1D, CD1E

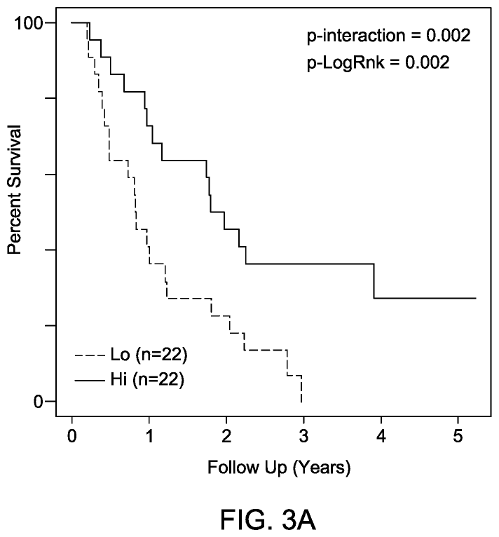

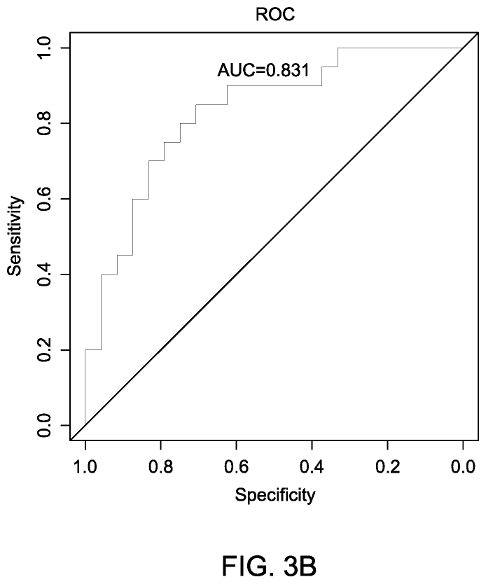

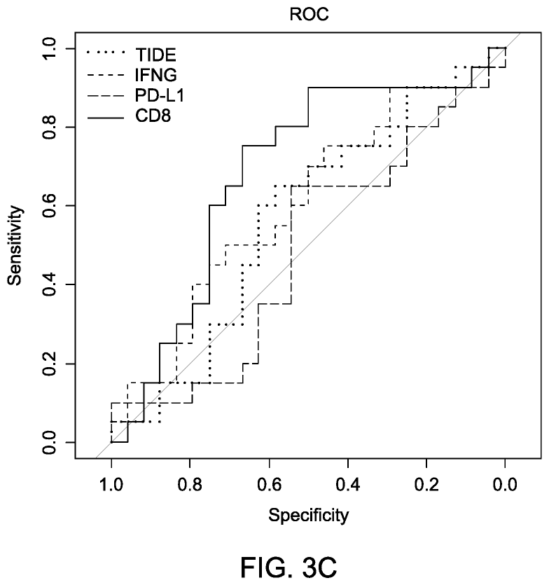

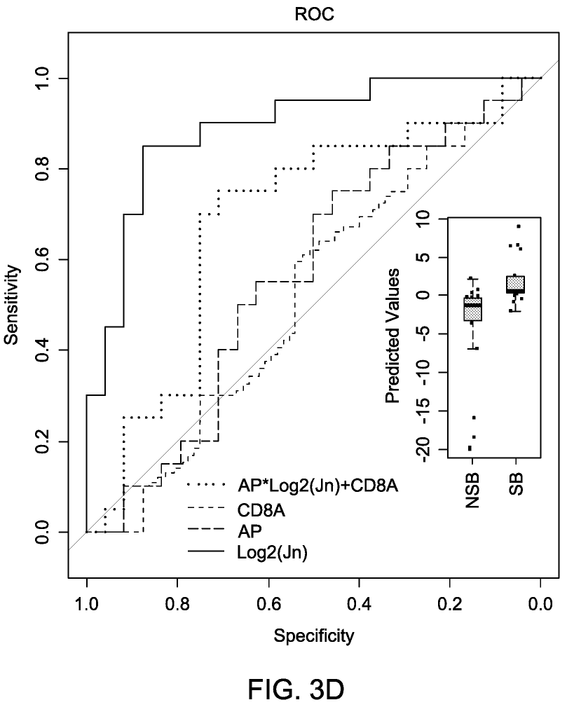

The interaction models that included the “REGULATION OF APP OF PEPTIDE ANTIGEN” gene set were further examined. Both the interaction parameter between this gene set and the junction burden, and the survival model were highly significant (Table 2 and FIG. 3A). Furthermore, this interaction was highly predictive of S_{1.5 yr} with an AUC of 0.831 (FIG. 3B). For comparison, several available gene models reported to associate with response to immune checkpoint inhibitors were tested including TIDE, IFNG, PD-L1, CD8, and others (Fu et al., Genome Med., 12:21 (2020)). In the present cohort, none of these other models performed as well as the interaction of APP gene sets with the junction burdens in predicting S_{1.5 yr}, but the most accurate of these gene models was the CD8 model with an AUC of 0.683 (FIG. 3C). Based on this observation, and to account for the role of antitumor lymphocytes in survival with immune checkpoint inhibition, CD8A was included in the prediction model. This addition increased the accuracy of the model from 0.831 to 0.890 (FIG. 3D).

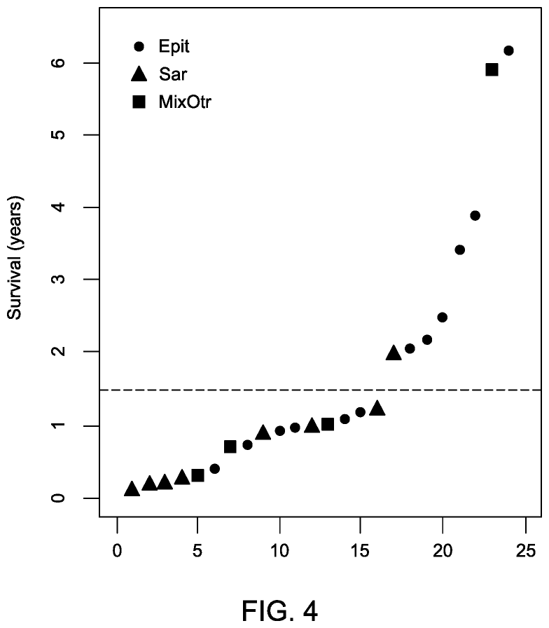

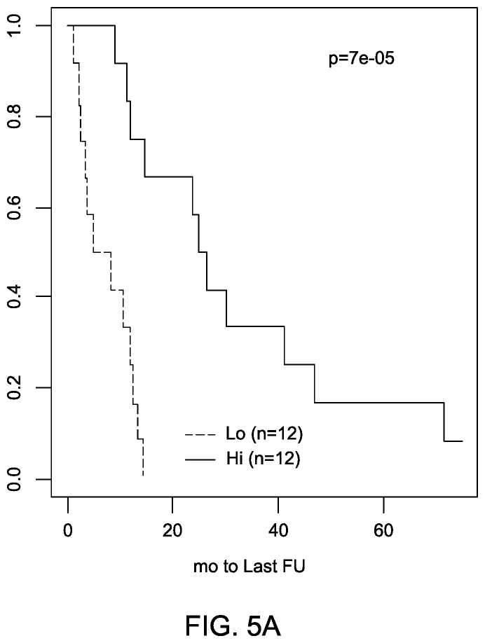

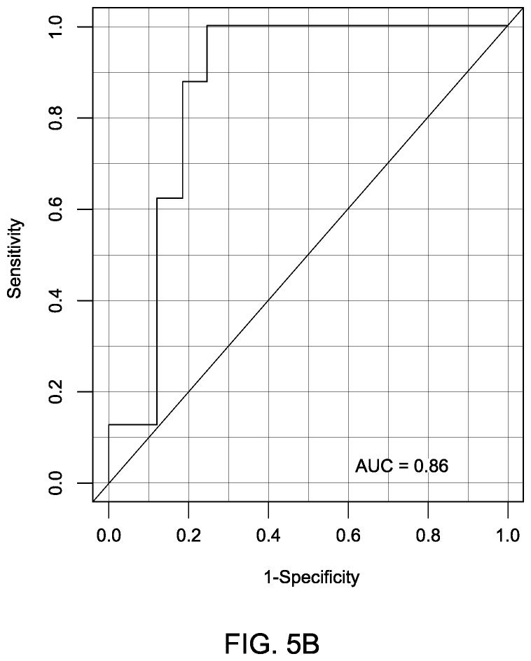

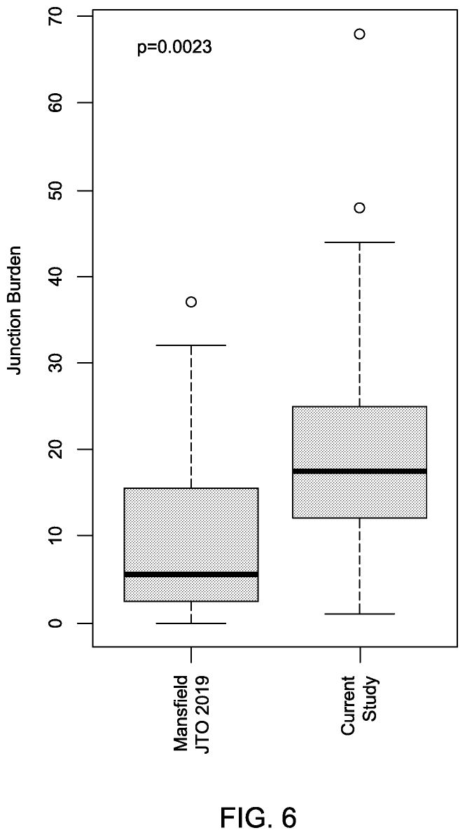

Finally, it was sought to determine if the interaction models were predictive of patient overall survival irrespective of treatment approach. A mesothelioma dataset that includes both chromosomal rearrangements from whole genome sequencing, and RNA-seq, is from a previous study of patients (n=24) who provided biopsy or surgical specimens prior to any cytotoxic systemic therapy (2019 cohort) (Mansfield et al., J. Thorac. Oncol., 14 (2): 276-287 (2019)). The patients in the 2019 cohort did not receive immune checkpoint inhibitors as these therapies were not available during their lifetimes. There was a break in overall survival at 1.5 years from diagnosis in this cohort that was used as the threshold for categorizing patients as NSB and SB (FIG. 4). The 2019 cohort performed similarly to other historic mesothelioma cohorts as a previously established mesothelioma survival signature gene set (Lopez-Rios et al., Cancer Res., 66:2970-2979 (2006)) had very high prognostic significance for both overall survival and S_{1.5 yr} (FIG. 5). No statistically significant interaction between the junction burden and any of the 12 APP gene sets on overall survival was found (Table 3). In further analysis, it was noted that the tumors in the 2019 cohort had fewer junctions than the current cohort (FIG. 6) which may have affected the predictive values of the interaction models.

TABLE 3
Antigen processing and presentation
gene set analysis in the 2019 cohort.

| | P-IA- | p-Log | P-IA- |
|---|---|---|---|
| | cox | Rank | lr |
| AP&P_OF_PEPTIDE_ANTIGEN_{—} | 0.24 | 0.44 | 0.08 |
| VIA_MHC_CLASS_IB | | | |
| AP&P_OF_PEPTIDE_ANTIGEN_{—} | 0.18 | 0.37 | 0.20 |
| VIA_MHC_CLASS_I | | | |
| AP&A_VIA_MHC_CLASS_IB | 0.25 | 0.48 | 0.20 |
| AP&P_OF_ENDOGENOUS_{—} | 0.13 | 0.31 | 0.10 |
| PEPTIDE_ANTIGEN | | | |
| AP&P_OF_ENDOGENOUS_{—} | 0.24 | 0.59 | 0.06 |
| PEPTIDE_ANTIGEN_VIA_MHC_{—} | | | |
| CLASS_I_VIA_ER_PATHWAY | | | |
| REGULATION_OF_AP&P | 0.37 | 0.52 | 0.38 |
| NEGATIVE_REGULATION_{—} | 0.46 | 0.54 | 0.36 |
| OF_AP&P | | | |
| POSITIVE_REGULATION_{—} | 0.33 | 0.51 | 0.44 |
| OF_AP&P | | | |
| REGULATION_OF_AP&P_{—} | 0.88 | 0.41 | 0.24 |
| OF_PEPTIDIE_ANTIGEN | | | |
| AP&P | 0.16 | 0.28 | 0.19 |
| AP&P_OF_ENDOGENOUS_{—} | 0.09 | 0.20 | 0.10 |
| ANTIGEN | | | |
| AP&P_OF_PEPTIDE_ANTIGEN | 0.17 | 0.31 | 0.17 |

The statistical significance of interactions (P-IA-cox) and log-rank (p-Log Rank) in cox models, interactions (P-IA-lr) and area under the curve (AUC) in logistic regression models are listed for the 2019 cohort.

In conclusion, in the context of antigen processing and presentation, junction burdens were associated with improved survival in patients with mesothelioma treated with immune checkpoint inhibitors. In the absence of antigen processing and presentation, junction burdens were associated with poor survival. The inclusion of genomic approaches that can detect structural variants, and transcriptomics to assess antigen processing and presentation, may help refine the selection of patients to receive immune checkpoint inhibitors.

### Example 2: Treating Pleural Mesothelioma

A biological sample (e.g., a pleural tissue sample) containing pleural mesothelioma cells is obtained from a human having pleural mesothelioma. The obtained sample is examined for the presence or absence of an elevated level of at least 6 different APP polypeptides.

If the presence of an elevated level of at least 6 different APP polypeptides is detected, then the human is identified as being likely to respond to one or more immune checkpoint inhibitors, and the human is administered one or more immune checkpoint inhibitors.

The administered one or more immune checkpoint inhibitors can reduce number of cancer cells within the human while improving the survival of the human.

### Example 3: Treating Pleural Mesothelioma

A biological sample (e.g., a pleural tissue sample) containing pleural mesothelioma cells is obtained from a human having pleural mesothelioma. The obtained sample is examined for the presence or absence of an elevated level of at least 6 different APP polypeptides.

If the absence of an elevated level of at least 6 different APP polypeptides is detected, then the human is identified as not being likely to respond to one or more immune checkpoint inhibitors, and the human is instead administered one or more alternative cancer treatments.

The administered alternative cancer treatments can reduce number of cancer cells within the human while improving the survival of the human.

### Example 4: Other Embodiments

Embodiment 1. A method for assessing a mammal having mesothelioma, wherein said mesothelioma comprises a high junction burden, and wherein said method comprises:
(a) determining if mesothelioma cells of said mammal contain the presence or absence of an elevated level of at least 6 APP polypeptides,
(b) classifying said mammal as being likely to respond to an immune checkpoint inhibitor if said presence is determined; and
(c) classifying said mammal as not being likely to respond to said immune checkpoint inhibitor if said absence is determined.

Embodiment 2. The method of embodiment 1, wherein said mammal is a human.

Embodiment 3. The method of any one of embodiments 1-2, wherein a tissue sample comprising said mesothelioma cells is used to determine if said mesothelioma cells contain said presence or absence.

Embodiment 4. The method of embodiment 3, wherein said tissue sample is a pleural tissue sample.

Embodiment 5. The method of any one of embodiments 1-4, wherein said high junction burden comprises at least 17 junctions per mb.

Embodiment 6. The method of any one of embodiments 1-5, wherein said mesothelioma is a pleural mesothelioma.

Embodiment 7. The method of any one of embodiments 1-6, wherein said APP polypeptides are selected from the group consisting of a PYCARD polypeptide, a HFE polypeptide, a HLA-DOA polypeptide, a HLA-DOB polypeptide, a TREM2 polypeptide, a TAPBPL polypeptide, a HLA-A polypeptide, a HLA-B polypeptide, a HLA-C polypeptide, a HLA-E polypeptide, a HLA-F polypeptide, a HLA-G polypeptide, a HLA-H polypeptide, a AZGP1 polypeptide, a B2M polypeptide, a TAP2 polypeptide, a ABCB9 polypeptide, a IDE polypeptide, a ERAP1 polypeptide, a ERAP2 polypeptide, a TAP1 polypeptide, a TAPBP polypeptide, a AP3B1 polypeptide, a AP3D1 polypeptide, a CD1A polypeptide, a CD1B polypeptide, a CD1C polypeptide, a CD1D polypeptide, a CD1E polypeptide, a ATG5 polypeptide, and a CD74 polypeptide.

Embodiment 8. The method of any one of embodiments 1-7, wherein said immune checkpoint inhibitor is selected from the group consisting of pembrolizumab, nivolumab, cemiplimab, atezolizumab, avelumab, durvalumab, and ipilimumab.

Embodiment 9. The method of any one of embodiments 1-8, wherein said method comprises determining said presence.

Embodiment 10. The method of any one of embodiments 1-8, wherein said method comprises determining said absence.

Embodiment 11. The method of any one of embodiments 1-8, wherein the presence or absence of said elevated level of at least one of said at least 6 APP polypeptides is determined by measuring mRNA encoding said at least one of said at least 6 APP polypeptides.

Embodiment 12. The method of any one of any one of embodiments 1-8, wherein the presence or absence of said elevated level of each of said at least 6 APP polypeptides is determined by measuring mRNA encoding said each of said at least 6 APP polypeptides.

Embodiment 13. A method for treating a mammal having mesothelioma, wherein said mesothelioma comprises a high junction burden, and wherein said method comprises:
(a) determining that mesothelioma cells of said mammal contain the presence of an elevated level of at least 6 APP polypeptides; and
(b) administering an immune checkpoint inhibitor to said mammal.

Embodiment 14. A method for treating a mammal having mesothelioma, wherein said mesothelioma comprises a high junction burden, and wherein said method comprises administering an immune checkpoint inhibitor to a mammal identified as having an elevated level of at least 6 APP polypeptides in mesothelioma cells of said mammal.

Embodiment 15. The method of any one of embodiments 13-14, wherein said mammal is a human.

Embodiment 16. The method of embodiment 13, wherein a tissue sample comprising said mesothelioma cells is used to determine that said mesothelioma cells contain said presence.

Embodiment 17. The method of embodiment 14, wherein a tissue sample comprising said mesothelioma cells was used to identify said mammal as having an elevated level of said at least 6 APP polypeptides.

Embodiment 18. The method of any one of embodiments 16 and 17, wherein said tissue sample is a pleural tissue sample.

Embodiment 19. The method of any one of embodiments 13-18, wherein said high junction burden comprises at least 17 junctions per mb.

Embodiment 20. The method of any one of embodiments 13-19, wherein said mesothelioma is a pleural mesothelioma.

Embodiment 21. The method of any one of embodiments 13-20, wherein said APP polypeptides are selected from the group consisting of a PYCARD polypeptide, a HFE polypeptide, a HLA-DOA polypeptide, a HLA-DOB polypeptide, a TREM2 polypeptide, a TAPBPL polypeptide, a HLA-A polypeptide, a HLA-B polypeptide, a HLA-C polypeptide, a HLA-E polypeptide, a HLA-F polypeptide, a HLA-G polypeptide, a HLA-H polypeptide, a AZGP1 polypeptide, a B2M polypeptide, a TAP2 polypeptide, a ABCB9 polypeptide, a IDE polypeptide, a ERAP1 polypeptide, a ERAP2 polypeptide, a TAP1 polypeptide, a TAPBP polypeptide, a AP3B1 polypeptide, a AP3D1 polypeptide, a CD1A polypeptide, a CD1B polypeptide, a CD1C polypeptide, a CD1D polypeptide, a CD1E polypeptide, a ATG5 polypeptide, and a CD74 polypeptide.

Embodiment 22. The method of any one of embodiments 13-21, wherein said immune checkpoint inhibitor is selected from the group consisting of pembrolizumab, nivolumab, cemiplimab, atezolizumab, avelumab, durvalumab, and ipilimumab.

Embodiment 23. The method of any one of embodiments 13-22, said method comprising administering to said mammal a cancer treatment selected from the group consisting of administering cisplatin, administering pemetrexed, administering carboplain, administering bevacizumab, administering vinorelbine, administering gemcitabine, administering ramucirumab, surgery, radiation therapy, tumor treatment fields, and cryotherapy.

Embodiment 24. The method of any one of embodiments 13 or 15-23, wherein the presence of said elevated level of at least one of said at least 6 APP polypeptides is determined by measuring mRNA encoding said at least one of said at least 6 APP polypeptides.

Embodiment 25. The method of any one of any one of 13 or 15-23, wherein the presence of said elevated level of each of said at least 6 APP polypeptides is determined by measuring mRNA encoding said each of said at least 6 APP polypeptides.

Embodiment 26. A method for treating a mammal having mesothelioma, wherein said mesothelioma comprises a high junction burden, and wherein said method comprises:
(a) determining if a sample from said mammal contains the absence of an elevated level of polypeptides expressed by at least 6 APP genes; and
(b) administering a cancer treatment to said mammal, wherein said cancer treatment is not an immune checkpoint inhibitor.

Embodiment 27. A method for treating a mammal having mesothelioma, wherein said mesothelioma comprises a high junction burden, and wherein said method comprises administering cancer treatment to a mammal identified as lacking an elevated level of polypeptides expressed by at least 6 APP genes in a sample obtained from said mammal, wherein said cancer treatment is not an immune checkpoint inhibitor.

Embodiment 28. The method of any one of embodiments 26-27, wherein said mammal is a human.

Embodiment 29. The method of embodiment 26, wherein a tissue sample comprising said mesothelioma cells is used to determine that said mesothelioma cells contain said absence.

Embodiment 30. The method of embodiment 27, wherein a tissue sample comprising said mesothelioma cells was used to identify said mammal as lacking an elevated level of said at least 6 APP polypeptides.

Embodiment 31. The method of embodiment 29 or embodiment 30, wherein said tissue sample is a pleural tissue sample.

Embodiment 32. The method of any one of embodiments 26-31, wherein said high junction burden comprises at least 17 junctions per mb.

Embodiment 33. The method of any one of embodiments 26-32, wherein said mesothelioma is a pleural mesothelioma.

Embodiment 34. The method of any one of embodiments 26-33, wherein said APP polypeptides are selected from the group consisting of a PYCARD polypeptide, a HFE polypeptide, a HLA-DOA polypeptide, a HLA-DOB polypeptide, a TREM2 polypeptide, a TAPBPL polypeptide, a HLA-A polypeptide, a HLA-B polypeptide, a HLA-C polypeptide, a HLA-E polypeptide, a HLA-F polypeptide, a HLA-G polypeptide, a HLA-H polypeptide, a AZGP1 polypeptide, a B2M polypeptide, a TAP2 polypeptide, a ABCB9 polypeptide, a IDE polypeptide, a ERAP1 polypeptide, a ERAP2 polypeptide, a TAP1 polypeptide, a TAPBP polypeptide, a AP3B1 polypeptide, a AP3D1 polypeptide, a CD1A polypeptide, a CD1B polypeptide, a CD1C polypeptide, a CD1D polypeptide, a CD1E polypeptide, a ATG5 polypeptide, and a CD74 polypeptide.

Embodiment 35. The method of any one of embodiments 26-34, wherein said cancer treatment comprises administering to the mammal a cancer drug selected from the group consisting of cisplatin, pemetrexed, carboplain, bevacizumab, vinorelbine, gemcitabine, and ramucirumab.

Embodiment 36. The method of any one of embodiments 26-34, wherein said cancer treatment comprises subjecting the mammal to a treatment selected from the group consisting of surgery, radiation therapy, tumor treatment fields, and cryotherapy.

Embodiment 37. The method of any one of embodiments 26 or 28-36, wherein the absence of said elevated level of at least one of said at least 6 APP polypeptides is determined by measuring mRNA encoding said at least one of said at least 6 APP polypeptides.

Embodiment 38. The method of any one of embodiments 26 or 28-36, wherein the absence of said elevated level of each of said at least 6 APP polypeptides is determined by measuring mRNA encoding said each of said at least 6 APP polypeptides.

### OTHER EMBODIMENTS

It is to be understood that while the invention has been described in conjunction with the detailed description thereof, the foregoing description is intended to illustrate and not limit the scope of the invention.

1-12. (canceled)

13. A method for treating a mammal having mesothelioma, wherein said mesothelioma comprises a high junction burden, and wherein said method comprises:
(a) determining that mesothelioma cells of said mammal contain the presence of an elevated level of at least 6 APP polypeptides; and
(b) administering an immune checkpoint inhibitor to said mammal.

14. A method for treating a mammal having mesothelioma, wherein said mesothelioma comprises a high junction burden, and wherein said method comprises administering an immune checkpoint inhibitor to a mammal identified as having an elevated level of at least 6 APP polypeptides in mesothelioma cells of said mammal.

15. The method of claim 13, wherein said mammal is a human.

16. The method of claim 13, wherein a tissue sample comprising said mesothelioma cells is used to determine that said mesothelioma cells contain said presence.

17. (canceled)

18. The method of claim 16, wherein said tissue sample is a pleural tissue sample.

19. The method of claim 13, wherein said high junction burden comprises at least 17 junctions per mb.

20. The method of claim 13, wherein said mesothelioma is a pleural mesothelioma.

21. The method of claim 13, wherein said APP polypeptides are selected from the group consisting of a PYCARD polypeptide, a HFE polypeptide, a HLA-DOA polypeptide, a HLA-DOB polypeptide, a TREM2 polypeptide, a TAPBPL polypeptide, a HLA-A polypeptide, a HLA-B polypeptide, a HLA-C polypeptide, a HLA-E polypeptide, a HLA-F polypeptide, a HLA-G polypeptide, a HLA-H polypeptide, a AZGP1 polypeptide, a B2M polypeptide, a TAP2 polypeptide, a ABCB9 polypeptide, a IDE polypeptide, a ERAP1 polypeptide, a ERAP2 polypeptide, a TAP1 polypeptide, a TAPBP polypeptide, a AP3B1 polypeptide, a AP3D1 polypeptide, a CD1A polypeptide, a CD1B polypeptide, a CD1C polypeptide, a CD1D polypeptide, a CD1E polypeptide, a ATG5 polypeptide, and a CD74 polypeptide.

22. The method of claim 13, wherein said immune checkpoint inhibitor is selected from the group consisting of pembrolizumab, nivolumab, cemiplimab, atezolizumab, avelumab, durvalumab, and ipilimumab.

23. The method of claim 13, said method comprising administering to said mammal a cancer treatment selected from the group consisting of administering cisplatin, administering pemetrexed, administering carboplain, administering bevacizumab, administering vinorelbine, administering gemcitabine, administering ramucirumab, surgery, radiation therapy, tumor treatment fields, and cryotherapy.

24. The method of claim 13, wherein the presence of said elevated level of at least one of said at least 6 APP polypeptides is determined by measuring mRNA encoding said at least one of said at least 6 APP polypeptides.

25. The method of claim 13, wherein the presence of said elevated level of each of said at least 6 APP polypeptides is determined by measuring mRNA encoding said each of said at least 6 APP polypeptides.

26. A method for treating a mammal having mesothelioma, wherein said mesothelioma comprises a high junction burden, and wherein said method comprises:
(a) determining if a sample from said mammal contains the absence of an elevated level of polypeptides expressed by at least 6 APP genes; and
(b) administering a cancer treatment to said mammal, wherein said cancer treatment is not an immune checkpoint inhibitor.

27. A method for treating a mammal having mesothelioma, wherein said mesothelioma comprises a high junction burden, and wherein said method comprises administering cancer treatment to a mammal identified as lacking an elevated level of polypeptides expressed by at least 6 APP genes in a sample obtained from said mammal, wherein said cancer treatment is not an immune checkpoint inhibitor.

28. The method of claim 26, wherein said mammal is a human.

29. The method of claim 26, wherein a tissue sample comprising said mesothelioma cells is used to determine that said mesothelioma cells contain said absence.

30. (canceled)

31. The method of claim 29, wherein said tissue sample is a pleural tissue sample.

32. The method of claim 26, wherein said high junction burden comprises at least 17 junctions per mb.

33. The method of claim 26, wherein said mesothelioma is a pleural mesothelioma.

34. The method of claim 26, wherein said APP polypeptides are selected from the group consisting of a PYCARD polypeptide, a HFE polypeptide, a HLA-DOA polypeptide, a HLA-DOB polypeptide, a TREM2 polypeptide, a TAPBPL polypeptide, a HLA-A polypeptide, a HLA-B polypeptide, a HLA-C polypeptide, a HLA-E polypeptide, a HLA-F polypeptide, a HLA-G polypeptide, a HLA-H polypeptide, a AZGP1 polypeptide, a B2M polypeptide, a TAP2 polypeptide, a ABCB9 polypeptide, a IDE polypeptide, a ERAP1 polypeptide, a ERAP2 polypeptide, a TAP1 polypeptide, a TAPBP polypeptide, a AP3B1 polypeptide, a AP3D1 polypeptide, a CD1A polypeptide, a CD1B polypeptide, a CD1C polypeptide, a CD1D polypeptide, a CD1E polypeptide, a ATG5 polypeptide, and a CD74 polypeptide.

35. The method of claim 26, wherein said cancer treatment comprises administering to the mammal a cancer drug selected from the group consisting of cisplatin, pemetrexed, carboplain, bevacizumab, vinorelbine, gemcitabine, and ramucirumab.

36. The method of claim 26, wherein said cancer treatment comprises subjecting the mammal to a treatment selected from the group consisting of surgery, radiation therapy, tumor treatment fields, and cryotherapy.

37. The method of claim 26, wherein the absence of said elevated level of at least one of said at least 6 APP polypeptides is determined by measuring mRNA encoding said at least one of said at least 6 APP polypeptides.

38. The method of claim 26, wherein the absence of said elevated level of each of said at least 6 APP polypeptides is determined by measuring mRNA encoding said each of said at least 6 APP polypeptides.
